(19) United States
(12) Patent Application Publication
(10) Pub. No.: US 2017/0049989 A1
(43) Pub. Date: Feb. 23, 2017

(54) METHODS AND KITS FOR TREATING DYSMOTILITY

(71) Applicant: Leonardo Kapural, Clemmons, NC (US)

(72) Inventor: Leonardo Kapural, Clemmons, NC (US)

(21) Appl. No.: 14/829,477

(22) Filed: Aug. 18, 2015

(51) Int. Cl. A61M 19/00; A61M 5/162; A61B 18/14; A61M 5/00; A61M 5/32; A61M 5/158; A61M 25/06

(52) U.S. Cl. CPC A61M 19/00; A61M 5/158; A61M 5/162; A61M 25/065; A61M 5/002; A61M 5/3286; A61B 18/1492; A61M 2202/048; A61M 2210/1003; A61B 2018/00577

(57) ABSTRACT

Described herein are methods and kits for treating dysmotility. Certain methods for treating dysmotility involve delivering a local anesthetic to at least one of the greater, lesser or least splanchnic nerves of a patient having dysmotility, or to the celiac plexus of the patient having dysmotility, to thereby reduce symptoms of dysmotility. Other method for treating dysmotility involve ablating a portion of at least one of the greater, lesser or least splanchnic nerves of a patient having dysmotility, to thereby reduce symptoms of dysmotility. Certain kits are for use in performing splanchnic nerve block or celiac plexus block to treat dysmotility. Other kits are for use in performing splanchnic nerve ablation to treat dysmotility.

### BACKGROUND

A person's gastrointestinal tract (which includes the esophagus, the stomach, the small intestines and the large intestines) typically has a rhythmic activity of muscle contractions, which is known as peristalsis. However, in persons having a motility disorder, which can also be referred to as dysmotility, this rhythm is disrupted, weakened or paralyzed, which may result in slow muscle contractions, fast muscle contractions or possibly no muscle contractions at all of the gastrointestinal tract. In other words, dysmotility refers to abnormal muscle contractions and/or dysfunctional nerves in the gastrointestinal tract, which causes slow transit or uncoordinated transit of food through the gastrointestinal tract.

The terms dysmotility and dysmotility syndrome, which are interchangeably used herein, are often used to generally refer to diseases of the muscles of the gastrointestinal tract in which the muscles do not work normally (hence the term dysmotility). Other more specific terms that are sometimes used to refer to types of dysmotility include gastroparesis (when the stomach is involved) and chronic intestinal pseudo-obstruction (when the intestines and the stomach are involved).

The terms dysmotility and dysmotility syndrome, as used herein, are intended to cover various different types of disorders relating to a person's gastrointestinal tract, including gastroparesis, functional dyspepsia, dumping syndrome, irritable bowel syndrome (IBS), cyclic vomiting syndrome, and esophageal dysmotility disorders. Eesophageal dysmotility disorders include, e.g., achalasia, diffuse esophageal spasm, hypertensive lower esophageal sphincter (LES), non-specific esophageal motility disorder, nutcracker esophagus, gastro-esophageal reflux disease (GERD), esophageal dysphagia, and functional chest pain. In addition, the terms dysmotility and dysmotility syndrome can also be used to refer to other dysmotility disorders of gastrointestinal tract like intestinal pseudoobstruction, intestinal dysmotility and short bowel syndrome.

Symptoms of dysmotility include nausea, an inability to digest solid foods, vomiting, abdominal bloating (also known as distention), abdominal pain, heartburn, gastroesophageal reflux, constipation and/or diarrhea. Persons with severe dysmotility may suffer from severe pain and/or malnutrition.

Existing treatments for dysmotility include the use of medications that attempt to stimulate intestinal motility and help with the propulsion of intestinal contents. Additionally, persons with dysmotility are often advised to modify their diets, e.g., to avoid foods that cause gas and/or are difficult to digest. Further, in order to ensure that adequate calories are consumed, persons with dysmotility often need to take meal supplements, and sometimes are admitted to hospitals for intravenous fluids and/or decompression of the intestines using a tube placed in the stomach.

In view of the discomfort and adverse effects that are caused by dysmotility, it would be beneficial if new treatments were available, especially for those persons who are nonresponsive to presently available treatments.

### SUMMARY

Described herein are methods and kits for treating dysmotility. Certain methods for treating dysmotility, according to certain embodiments of the present invention, involve delivering a local anesthetic to at least one of the greater, lesser or least splanchnic nerves of a patient having dysmotility, or to the celiac plexus of the patient having dysmotility, to thereby reduce symptoms of dysmotility. More specifically, in accordance with certain embodiments, the local anesthetic is delivered bilaterally to the at least one of the greater, lesser or least splanchnic nerves of the patient to achieve bilateral splanchnic nerve block, to thereby reduce symptoms of dysmotility. In such embodiments, the local anesthetic (delivered to at least one of the greater, lesser or least splanchnic nerves) interrupts efferent sympathetic fiber transmission from the splanchnic nerves of the patient to the spinal cord and brain of the patient.

In accordance with certain embodiments, the local anesthetic includes at least one of bupivacaine, chloroprocaine, mepivacaine, ropivacaine, or lidocaine. For a more specific example, the local anesthetic includes bupivacaine and has a concentration of bupivacaine between 0.25% and 0.75%, inclusive.

In accordance with an embodiment, a method includes inserting a portion of a needle into the patient having dysmotility, positioning a distal end of the needle adjacent to at least one of the thoracic vertebral segment numbers 11 or 12 (T11 or T12) of the patient. Then, while the distal end of the needle is positioned adjacent to the at least one of the T11 or T12 of the patient, the needle is used to deliver the local anesthetic to at least one of the greater, lesser or least splanchnic nerves of the patient, to thereby reduce symptoms of dysmotility.

In accordance with another embodiment, a method includes inserting a portion of a needle into the patient having dysmotility, positioning a distal end of the needle adjacent to the L1 lumbar vertebrae of the patient, and inserting a catheter through the needle and passing a distal end of the catheter past the distal end of the needle so that the distal end of the catheter is adjacent to least one of the thoracic vertebral segment numbers 11 or 12 (T11 or T12) of the patient. Then, while the distal end of the catheter is positioned adjacent to the at least one of the T11 or T12 of the patient, the catheter is used to deliver the local anesthetic to at least one of the greater, lesser or least splanchnic nerves of the patient, to thereby reduce symptoms of dysmotility.

In accordance with specific embodiments, the local anesthetic is delivered bilaterally to the celiac plexus of a patient of to achieve bilateral celiac plexus block, to thereby reduce symptoms of dysmotility. In such embodiments, the local anesthetic, delivered to the celiac plexus of the patient, interrupts efferent sympathetic fiber transmission from celiac plexus of the patient to the splanchnic nerves, spinal cord and brain of the patient.

In accordance with an embodiment, a method includes inserting a portion of a needle into the patient having dysmotility, and positioning a distal end of the needle adjacent to the L1 lumbar vertebrae of the patient. Then, while the distal end of the needle is positioned adjacent to the L1 lumbar vertebrae of the patient, the needle is used to deliver the local anesthetic to the celiac plexus of the patient, to thereby reduce symptoms of dysmotility.

In accordance with an alternative embodiment, a method includes inserting a portion of a needle into the patient having dysmotility, positioning a distal end of the needle posterior to and between a mid to anterior portion of the L1 lumbar vertebral body, and inserting a catheter through the needle and passing a distal end of the catheter past the distal end of the needle so that the distal end of the catheter is adjacent to the L1 lumbar vertebrae of the patient. Then, while the distal end of the catheter is positioned adjacent to the L1 lumbar vertebrae of the patient, the catheter is used to deliver the local anesthetic to the celiac plexus of the patient, to thereby reduce symptoms of dysmotility.

Certain embodiments for treating dysmotility involve ablating a portion of at least one of the greater, lesser or least splanchnic nerves of a patient having dysmotility, to thereby reduce symptoms of dysmotility. Such ablating, which is preferably performed bilaterally, can be performed using radiofrequency ablation. In accordance with an embodiment, a method includes inserting a portion of a needle into the patient having dysmotility, wherein a proximal portion of the needle is insulated and a distal portion of the needle is uninsulated. The uninsulated distal end of the needle is positioned adjacent to one of the thoracic vertebral segment numbers 11 or 12 (T11 or T12) of the patient. Then, while the uninsulated distal end of the needle is positioned adjacent to one of the T11 or T12 of the patient, the uninsulated distal end of the needle is used to deliver an alternating radiofrequency (RF) current to at least one of the greater, lesser or least splanchnic nerves of the patient having dysmotility. In accordance with certain embodiments, a frequency of the alternating RF current is within a range of 450 kHz to 1200 kHz, inclusive.

In accordance with other embodiments, alternative types of ablation techniques are used to perform splanchnic nerve ablation to treat dysmotility. For example, the ablating can alternatively be performing using electromagnetic ablation, ultrasound ablation, microwave ablation, infrared light ablation, hot water ablation, or cryogenic cooling ablation, but is not limited thereto.

Certain embodiments of the present invention are directed to kits for use in performing splanchnic nerve block or celiac plexus block to treat dysmotility. Such a kit can include a pair of spinal needles each having a length between 5 and 7 inches, inclusive, and an outer diameter between 18 and 25 gauge, inclusive. Additionally, the kit can include a first vial storing a first local anesthetic (e.g., lidocaine) for use in anesthetizing (i.e., numbing) a patient's skin. The kit can also include an injection needle having a length between 1 and 2 inches, inclusive, and an outer diameter between 22 and 27 gauge, inclusive, wherein the injection needle is for use in injecting portions of the first local anesthetic at sites where the spinal needles are to be inserted into a patient's skin. The kit can further include a first syringe for use in injecting portions of the first local anesthetic through the injection needle. The kit can additionally include a second vial storing a second local anesthetic that is to be delivered, using the pair of spinal needles, to at least one of the greater, lesser or least splanchnic nerves of a patient or to the celiac plexus of the patient. The second local anesthetic can include bupivacaine, chloroprocaine, mepivacaine, ropivacaine, and/or lidocaine, but is not limited thereto. Further, the kit can include a second syringe for use in injecting portions of the second local anesthetic through the pair of spinal needles. The second syringe can be configured to connect to distal ends of the pair of spinal needles. The reason that a pair of (i.e., two) spinal needles are included in the kit is so that the splanchnic nerve block or celiac plexus block can be performed bilaterally.

In accordance with certain embodiments, the kit can also include a catheter for use in threading through each of the pair of spinal needles and delivering portions of the second local anesthetic to at least one of the greater, lesser or least splanchnic nerves or the celiac plexus of a patient. In such embodiments, the catheter has a length that is greater than the length of each of the spinal needles, so that while the catheter is threaded through one of the spinal needles, a proximal end of the catheter can extend from a proximal end of the one of the spinal needles while a distal end of the catheter extends from a distal end of the one of the spinal needles. An outer diameter of the catheter is smaller than an inner diameter of each of the spinal needles so that the catheter can be threaded through each of the spinal needles. The second syringe can be configured to connect to the distal end of the catheter. The kit can also include an antiseptic and one or more sponges for use in cleaning portions of a patient's skin prior to the needles being injected therein. Further, the kit can also include a vial storing a saline solution for use in diluting a concentration of at least one of the first and second local anesthetics.

Alternative kits of embodiments of the present invention are for use in performing splanchnic nerve ablation to treat dysmotility. Such a kit can include a pair of active tip radiofrequency (RF) needles each having a total length between 5 and 7 inches, inclusive, and an outer diameter between 18 and 25 gauge, inclusive, wherein an outer surface of each the RF needles is insulated except an uninsulated distal portion that is between 0.25 and 0.75 inches in length. The remaining components of this alternative kit can be the same as in the kit summarized above.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is an elevation view of the thoracic splanchnic nerves, the celiac plexus, and related anatomical structures.

FIG. 2 is a high level flow diagram that is used to summarize methods for treating dysmotility using splanchnic nerve block, according to specific embodiments of the present invention.

FIG. 3 is a high level flow diagram that is used to summarize methods for treating dysmotility using celiac plexus block, according to specific embodiments of the present invention.

FIG. 4 is a high level flow diagram that is used to summarize methods for treating dysmotility using splanchnic nerve ablation, according to specific embodiments of the present invention.

FIG. 5A illustrates a kit, according to an embodiment, for use in performing splanchnic nerve block or celiac plexus block to treat dysmotility.

FIG. 5B illustrates a kit, according to another embodiment, for use in performing splanchnic nerve block or celiac plexus block to treat dysmotility.

FIG. 5C illustrates a kit, according to an embodiment, for use in performing splanchnic nerve ablation to treat dysmotility.

### DETAILED DESCRIPTION

Certain embodiments of the present invention, which are described below, relate to treating dysmotility using splanchnic nerve block, splanchnic nerve radiofrequency ablation and/or celiac plexus block. However, prior to providing details of such embodiments, it is first useful to describe certain portions of the human anatomy that are relevant to the embodiments described herein.

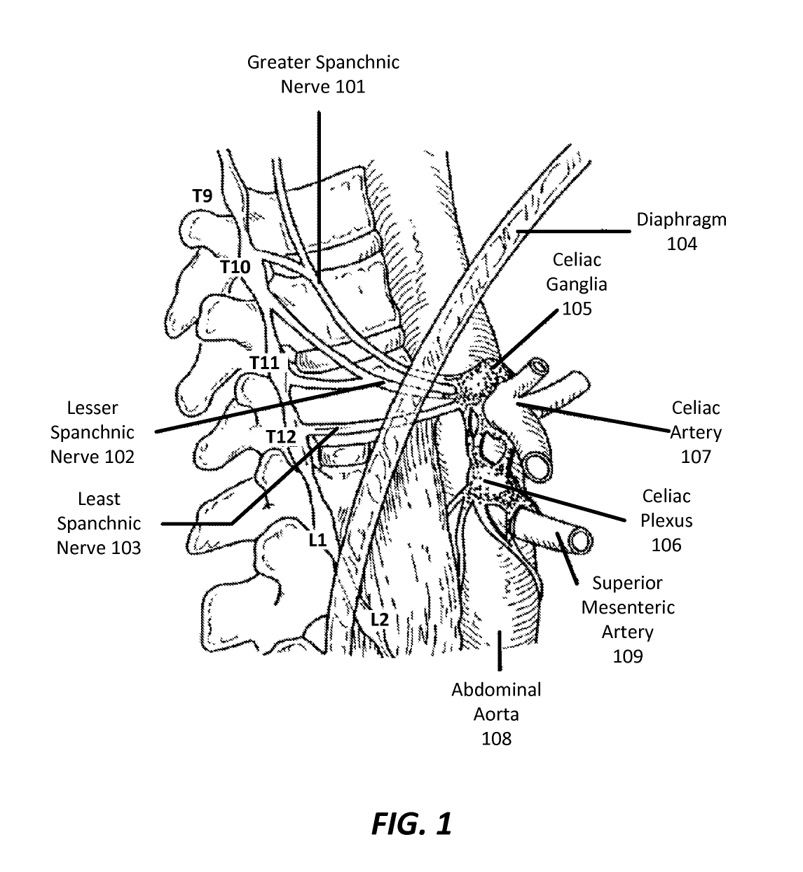

FIG. 1 is an elevation view of the thoracic splanchnic nerves, the celiac plexus, and related anatomical structures. The thoracic splanchnic nerves include greater, lesser and least splanchnic nerves, labeled 101, 102 and 103, respectively, which arise from preganglionic fibers that course through the crura of the diaphragm 104 before synapsing in the celiac ganglia 105. Thoracic splanchnic nerves travel inferiorly to provide sympathetic innervation to the abdomen. The thoracic splanchnic nerves contain preganglionic sympathetic and general visceral afferent fibers.

The greater splanchnic nerve 101 travels through the diaphragm 104 and enters the abdominal cavity, where its fibers synapse at the celiac ganglia 105. The greater splanchnic nerve 101 contributes to the celiac plexus 106. The celiac plexus 106 is a network of nerves located in the vicinity of where the celiac artery 107 (also known as the celiac trunk) branches from the abdominal aorta 108. Fibers in the greater splanchnic nerve 101 modulate the activity of the enteric nervous system of the foregut, and also provide the sympathetic innervation to the adrenal medulla, stimulating catecholamine release. The lesser splanchnic nerve 102 travels inferiorly, lateral to the greater splanchnic nerve 101. Fibers in the lesser splanchnic nerve 102 synapse with their postganglionic counterparts in the superior mesenteric ganglia, or in the aorticorenal ganglion. The lesser splanchnic nerve 102 modulates the activity of the enteric nervous system of the midgut. The least splanchnic nerve 103 travels into the abdomen, where its fibers synapse in the renal ganglia.

Cadaveric studies have revealed that the greater splanchnic nerve 101 originates between thoracic vertebral segment numbers 5 through 10 (T5-T10), while the lesser splanchnic nerve 102 arises from thoracic vertebral segment numbers 9 and 10 (T9-T10), and the least splanchnic nerve 103 arises from thoracic vertebral segment numbers 11 and 12 (T11-T12). Interventional approaches target these nerves at either T11 or T12, or at both T11 and T12 when the splanchnic nerves are in close proximity to the vertebral body.

Also shown in FIG. 1 are the celiac artery 107 and the celiac plexus 106. The celiac artery 107, which branches from the abdominal aorta 108 at the thoracic vertebral segment number 12 (T12), supplies oxygenated blood to the liver, stomach, abdominal esophagus, spleen and the superior half of both the duodenum and the pancreas. The celiac plexus 106 (which is also known as the coeliac plexus, and as the solar plexus) is a complex network of nerves (a nerve plexus) located in the abdomen, where the celiac artery 107, superior mesenteric artery 109, and renal arteries branch from the abdominal aorta 108. The celiac plexus 106 is located behind the stomach and the omental bursa, and in front of the crura of the diaphragm 104, on the level of the first lumbar vertebra (L1).

The diaphragm 104 shown in FIG. 1, which is also knows as the thoracic diaphragm, is a sheet of internal skeletal muscle hat extends across the bottom of the thoracic cavity. The diaphragm 104 separates the thoracic cavity containing the heart and lungs, from the abdominal cavity and performs an important function in respiration, wherein as the diaphragm 104 contracts, the volume of the thoracic cavity increases and air is drawn into the lungs. The celiac ganglia 105 shown in FIG. 1, which are also known as the coeliac ganglia, are two large irregularly shaped masses of nerve tissue in the upper abdomen that are part of the sympathetic subdivision of the autonomic nervous system and innervate most of the digestive tract.

### Splanchnic Nerve Block to Treat Dysmotility

In accordance with specific embodiments of the present invention, a local anesthetic is used to achieve splanchnic nerve block to treat dysmotility. Beneficially, such embodiments offer the ability to both avoid the celiac artery and selectively interrupt efferent sympathetic fiber transmission before the splanchnic nerves reach the celiac plexus. Such embodiments, which are discussed in additional detail below, are referred to as splanchnic nerve block embodiments, or simply splanchnic nerve block.

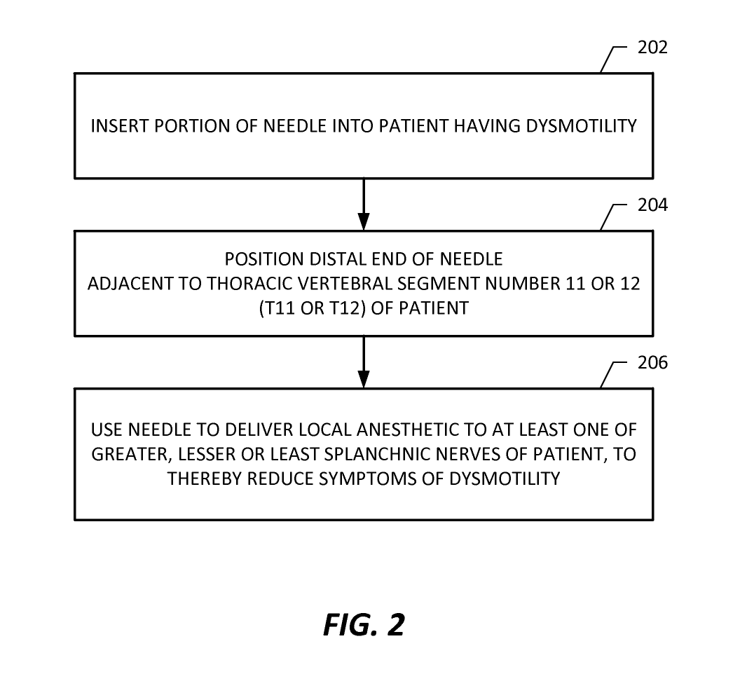

FIG. 2 is a high level flow diagram that is used to summarize methods for treating dysmotility using splanchnic nerve block. Referring to FIG. 2, step 202 involves inserting a portion of a needle into the patient having dysmotility. Step 204 involves positioning a distal end of the needle adjacent to one of the thoracic vertebral segment numbers 11 or 12 (T11 or T12) of the patient. While the distal end of the needle is positioned adjacent to the T11 or T12 of the patient, step 206 involves using the needle to deliver a local anesthetic to at least one of the greater, lesser or least splanchnic nerves of the patient, to thereby reduce symptoms of dysmotility. Step 206 can include attaching a syringe, which includes the local anesthetic, to a proximal end of the needle, and using the syringe to inject the local anesthetic to at least one of the greater, lesser or least splanchnic nerves of the patient. In accordance with specific embodiments, steps 202, 204 and 206 are performed twice, once from the left side of the spine and once from the right side of the spine, to achieve bilateral splanchnic nerve block. Additional details of these steps are discussed below.

The local anesthetic, which is delivered to at least one of the greater, lesser or least splanchnic nerves, interrupts efferent sympathetic fiber transmission from the splanchnic nerves of the patient to the spinal cord and brain of the patient. In accordance with an embodiment, the local anesthetic includes bupivacaine and has a concentration of bupivacaine between 0.25% and 0.75%, inclusive. Alternative or additional types of local anesthetic can be used to perform splanchnic nerve block to treat dysmotility, such as, but not limited to, chloroprocaine (having a concentration between 1% and 2%, inclusive), mepivacaine (having a concentration between 0.25% and 0.5%, inclusive), ropivacaine (having a concentration between 0.25% and 0.5%, inclusive), or lidocaine (having a concentration between 1% and 2%, inclusive), or combinations thereof.

In accordance with an embodiment, steps 202 and 204 are performed using a dorsal approach under fluoroscopy or ultrasound guidance, or some other medical imaging technique. For a more specific example, the dorsal approach can be performed under fluoroscopy by positioning the patient prone and identifying the T11 or T12 vertebral bodies using radiographic imaging. Alternatively ultrasound guidance can be used. After squaring the vertebral end plates and introducing a 10 to 15-degree ipsilateral oblique fluoroscopic angle, the junction of the ribs and the vertebral body will become visible. A caudal view is preferred since it exposes more of the concavity of the target vertebral body T11 or T12. In accordance with an embodiment, the aforementioned junction of the ribs and the vertebral body is the landmark where the needle will be placed through the skin at step 202.

More specifically, following aseptic preparation of the injection site, e.g., using a povidone-iodine solution, a local anesthetic skin wheal is created and a spinal needle is inserted through the patient's skin and advanced coaxially hugging the lateral aspect of the mid portion of the T11 or T12 vertebral bodies. In accordance with certain embodiments, the local anesthetic skin wheal can be created by delivering lidocaine having a concentration between 0.5% and 2% just below the skin using a needle having a length between 1 and 2 inches, inclusive, and a gauge between 22 and 27, inclusive. The spinal needle preferably has a length between 5 and 7 inches, inclusive, and a gauge between 18 and 25, inclusive, and more preferably a gauge between 18 and 22, inclusive. The needle trajectory preferably courses along the mid-portion of the T11 or T12 vertebral body to avoid inadvertent entry into a disc or eliciting paresthesia along a nerve root. A cross-table lateral fluoroscopic image can be used to reveal the needle advancing ventrally to lie at the middle third of the T11 or T12 vertebral body. In accordance with an embodiment, 1 to 3 milliliters of a radio-opaque contrast can be injected using the spinal needle 512 used to demonstrate spread posterior to the aorta and anterior to the foramen. For splanchnic nerve block performed at the T11 or T12 level, because of the proximity to the pleura, the final needle position preferably does not extend beyond the anterior third of the T11 or T12 vertebral body.

In accordance with alternative embodiments, instead of positioning the distal end of a spinal needle adjacent to one of the thoracic vertebral segment numbers 11 or 12 (T11 or T12) of the patient, the distal end of the needle can instead be positioned adjacent to the L1 lumbar vertebrae of the patient. A flexible catheter (e.g., a nylon catheter) can then be inserted through the needle, and a distal end of the catheter can be extended past the distal end of the needle so that the distal end of the catheter is adjacent to one of thoracic vertebral segment numbers 11 or 12 (T11 or T12) of the patient. A syringe can then be attached to a proximal end the catheter and the local anesthetic (examples of which were discussed above) can be injected through the catheter to thereby be delivered to at least one of the greater, lesser or least splanchnic nerves of the patient, to thereby reduce symptoms of dysmotility.

In accordance with certain embodiments, between steps 204 and 206, a non-ionic radio-opaque contrast can be injected through the needle to check that the needle is extravascular and has not punctured a pleural cavity. More specifically, where the needle is positioned extravascular and has not punctured a pleural cavity, the contrast injectate should be visible at the injection site and show little spread when viewed using fluoroscopy or some other medical imaging. However, where the needle is positional intravascular, which is undesirable, the contrast will be seen only very briefly, due to vessel outflow, or not seen at all if the flow of the vessel is too fast for the contrast to be observed. Where the needle has punctured a pleural cavity, which is also undesirable, the contrast injectate will show spread when viewed using fluoroscopy or some other medical imaging. If it is determined that the needle is positioned intravascularly, or has punctured a pleural cavity, the distal end of the needle should be repositioned and retested using the contrast before the needle is used to deliver the local anesthetic used to perform splanchnic nerve block. In the rare situation where a needle punctures one of the patient's two pleural cavities, the splanchnic nerve block should not be performed on the other side of the spine, to complete the bilateral splanchnic nerve block, until after the punctured plural cavity has had time to heal, because bilateral pneumothorax can be life threatening.

### Celiac Plexus Block to Treat Dysmotility

In accordance with other embodiments of the present invention, a local anesthetic is delivered to the celiac plexus to treat dysmotility. Such embodiments, which are discussed in additional detail below, are referred to as celiac plexus block embodiments, or simply celiac plexus block.

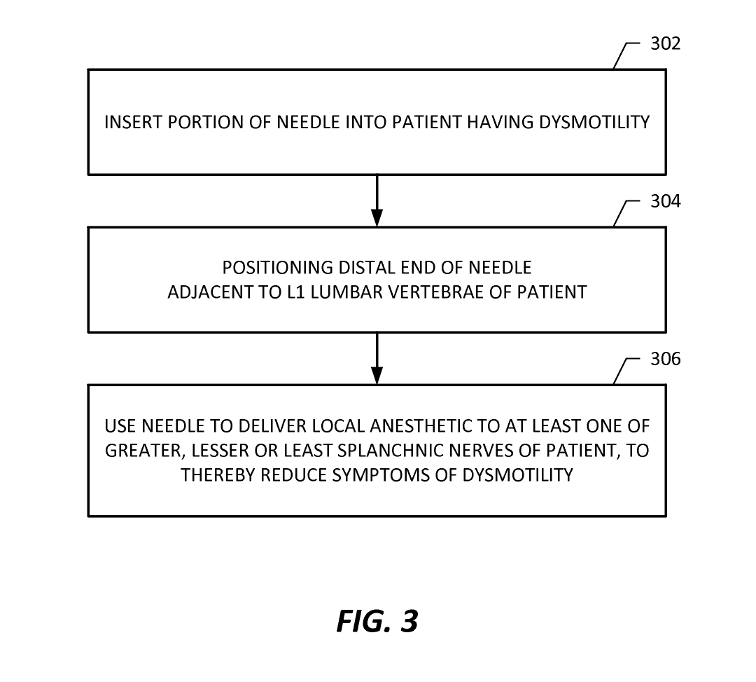

FIG. 3 is a high level flow diagram that is used to summarize methods for treating dysmotility using celiac plexus block, according to specific embodiments of the present invention. Referring to FIG. 3, step 302 involves inserting a portion of a needle into the patient having dysmotility. Step 304 involves positioning a distal end of the needle adjacent to the L1 lumbar vertebrae of the patient. The L1 lumbar vertebrae is also known as, and referred to herein as, the L1 vertebral body. While the distal end of the needle is positioned adjacent to the L1 lumbar vertebrae of the patient, step 306 involves using the needle to deliver a local anesthetic to the celiac plexus of the patient, to thereby reduce symptoms of dysmotility. Step 306 can include attaching a syringe, which includes the local anesthetic, to a proximal end of the needle, and using the syringe to inject the local anesthetic to the celiac plexus. In accordance with specific embodiments, steps 302, 304 and 306 are performed twice, once from the left side of the spine and once from the right side of the spine, to achieve bilateral celiac plexus nerve block. Additional details of these steps are discussed below.

The local anesthetic, which is delivered to the celiac plexus of the patient, interrupts efferent sympathetic fiber transmission from celiac plexus of the patient to the splanchnic nerves, spinal cord and brain of the patient. In accordance with an embodiment, the local anesthetic includes bupivacaine and has a concentration of bupivacaine between 0.25% and 0.75%, inclusive. Alternative or additional types of local anesthetic can be used to perform celiac plexus block to treat dysmotility, such as, but not limited to, chloroprocaine (having a concentration between 1% and 2%, inclusive), mepivacaine (having a concentration between 0.25% and 0.5%, inclusive), ropivacaine (having a concentration between 0.25% and 0.5%, inclusive), or lidocaine (having a concentration between 1% and 2%, inclusive), or combinations thereof.

The celiac plexus can be approached with via a dorsal retro or an anterocrural approach. In accordance with an embodiment, after positioning the patient prone and visualizing the L1 vertebral body and the left L1 transverse process, a skin wheal is created approximately 2.5 inches (i.e., approximately 6 centimeters) from the midline. More specifically, following antiseptic preparation of the injection site, e.g., using a povidone-iodine solution, a local anesthetic skin wheal is created and a spinal needle is inserted through the patient's skin and advanced coaxially hugging the lateral aspect of the mid portion of the T11 or T12 vertebral bodies. In accordance with certain embodiments, the local anesthetic skin wheal can be created by delivering lidocaine having a concentration between 0.5% and 2% just below the skin using a needle having a length between 1 and 2 inches, inclusive, and a gauge between 22 and 27, inclusive. The spinal needle preferably has a length between 5 and 7 inches, inclusive, and a gauge between 18 and 25, inclusive, and more preferably a gauge between 18 and 22, inclusive.

In accordance with an embodiment, the spinal needle is advanced ventrally avoiding the transverse process and coursing adjacent to the mid-portion of the L1 vertebral body. In accordance with an embodiment, an injection of 1 to 3 milliliters of a radio-opaque contrast can be used to demonstrate cranio-caudal spread and excludes vascular uptake. In accordance with an embodiment, the needle is advanced to terminate just beyond the anterior aspect of the L1 vertebral body behind the posterior wall of the aorta. If the contrast spread is unilateral as visualized by an antero-posterior fluoroscopic image, the procedure may need to be repeated via an identical approach on the contralateral side of the L1 vertebral body.

Increasing the volume of local anesthetic injectate is one way to account for anatomic variations in the takeoff of the celiac artery along the cranio-caudal axis if the procedure is performed with fluoroscopy rather than computed tomography (CT).

Alternative techniques for delivering a local anesthetic to the celiac plexus are possible, and within the scope of embodiments of the present invention. For example, various dorsal or ventral approaches that employ endoscopic ultrasound, fluoroscopy, computed tomography (CT) or magnetic resonance imaging (MRI) guidance for the needle placement can be used. Surgical approaches may alternatively be used, which include thoracoscopic and open approaches via laparotomy.

In accordance with alternative embodiments, instead of positioning the distal end of a spinal needle adjacent to the L1 lumbar vertebrae of the patient, the distal end of the needle can instead be positioned posterior to and between a mid to anterior portion of the L1 lumbar vertebral body, and then a flexible catheter (e.g., a nylon catheter) can be inserted through the needle, and a distal end of the catheter can be extended past the distal end of the needle so that the distal end of the catheter is adjacent to the L1 lumbar vertebrae of the patient the patient. A syringe can then be attached to a proximal end the catheter and the local anesthetic (examples of which were discussed above) can be injected through the catheter to thereby be delivered to the celiac plexus of the patient, to thereby reduce symptoms of dysmotility.

In accordance with certain embodiments, between steps 304 and 306, a non-ionic radio-opaque contrast can be injected through the needle to check that the needle is extravascular and has not punctured a pleural cavity. If it is determined that the needle is positioned intravascularly, or has punctured a pleural cavity, the distal end of the needle should be repositioned and retested using the contrast before the needle is used to deliver the local anesthetic used to perform celiac plexus block. In the rare situation where a needle punctures one of the patient's two pleural cavities, the celiac plexus block should not be performed on the other side of the spine, to complete the bilateral celiac plexus block, until after the punctured plural cavity has had time to heal.

### Splanchnic Nerve Ablation to Treat Dysmotility

At the level of the lower thoracic vertebral bodies, the splanchnic nerves have a predictable course bordered laterally by the pleura, ventrally by the posterior mediastinum, medially by the thoracic vertebral bodies and dorsally by the pleural attachments to the vertebra. Additionally, this location is amenable to radiofrequency neurotomy (also known as radiofrequency ablation) of one or more of the greater, lesser or least splanchnic nerves to treat dysmotility. Other types of ablation techniques can alternatively be used. Such embodiments, which are discussed in additional detail below, are referred to as splanchnic nerve ablation. Splanchnic nerve ablation may be used, for example, with those patients who experience temporary but non-sustained relief from splanchnic nerve block or celiac plexus block that utilizes a local anesthetic, which techniques were described above with reference to FIGS. 2 and 3.

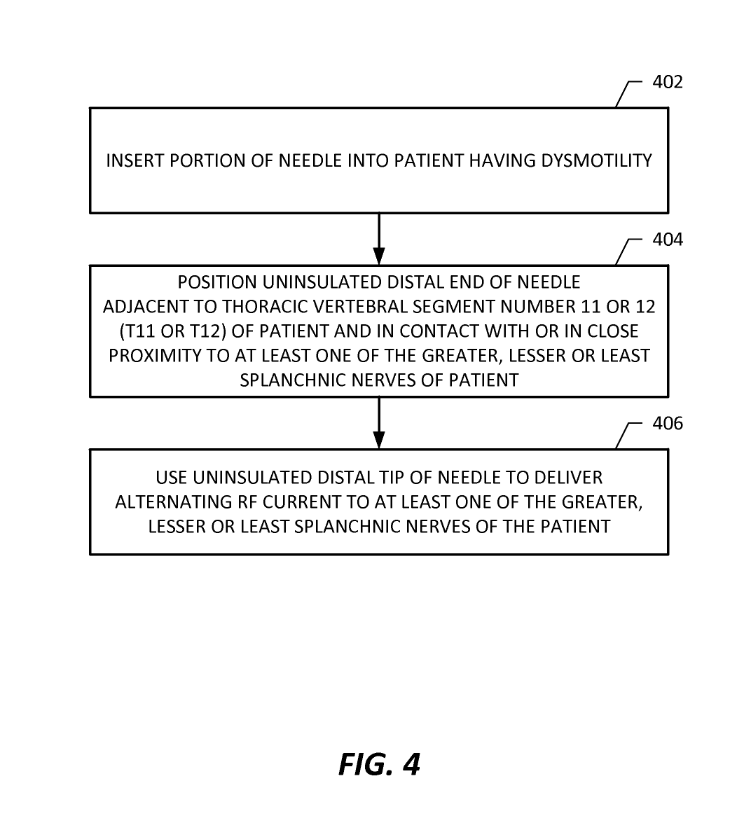

FIG. 4 is a high level flow diagram that is used to summarize methods for treating dysmotility using splanchnic nerve ablation, according to specific embodiments of the present invention. Referring to FIG. 4, step 402 involves inserting a portion of a needle into the patient having dysmotility, wherein a proximal portion of the needle is insulated and a distal portion of the needle is uninsulated. In accordance with specific embodiments of the present invention, the needle inserted at step 402 is an active tip radiofrequency (RF) needle having an outer surface that is insulated except an uninsulated distal tip portion. Step 404 involves positioning the uninsulated distal end of the needle adjacent to at least one of thoracic vertebral segment numbers 11 or 12 (T11 or T12) of the patient and in contact with or in close proximity to (e.g., within 5 mm) at least one of the greater, lesser or least splanchnic nerves of the patient having dysmotility. The uninsulated distal tip of the needle can be positioned in contact with or close proximity to its target, i.e., at least one of the greater, lesser or least splanchnic nerves, using nerve stimulation and/or image guidance. Step 406 involves using the uninsulated distal tip of the needle to deliver an alternating radiofrequency (RF) current to the at least one of the greater, lesser or least splanchnic nerves of the patient having dysmotility. The alternating RF current used to perform RF ablation can be generated by a commercially available RF generator (which can also be referred to as an RF machine, or an RF ablation generator), which is coupled, via a cable, to the proximal end of the needle used to deliver the RF current to the target tissue.

In accordance with specific embodiments, the frequency of the alternating RF current is within the range of 450 kHz to 1200 kHz, inclusive. The use of other frequencies are also possible and within embodiments of the present invention. Since the embodiments described above with reference to FIG. 4 utilize RF ablation, such embodiments can more specifically be referred to as splanchnic nerve radiofrequency ablation.

In accordance with certain embodiments, the positioning of the needle used to perform splanchnic nerve radiofrequency ablation is similar to the positioning of the needle used to perform splanchnic nerve block. In other words, steps 402 and 404 can be performed using the same or similar techniques to those described above with reference to steps 202 and 204 in FIG. 2.

In accordance with certain embodiments, step 404 includes positioning the uninsulated distal end of the needle parallel to the target so at step 406 the RF energy coagulates transversely. A RF pulse generator, to which a proximal end of the needle is connected via a cable, produces an electric field concentrated at the uninsulated distal end of the needle. The transfer of energy generates molecular oscillations that produce ionic friction and heat within the tissues. Once the cells are heated above a certain temperature, controlled tissue destruction occurs causing a lesion surrounding the uninsulated tip.

In accordance with specific embodiments, as part of step 404, or between steps 404 and 406, prior to performing ablation, the RF needle is used to deliver stimulation pulses to test for a sensory response. For example, stimulation pulses having a relatively low frequency, e.g., 50 Hz, and a pulse amplitude of about 0.5 to 1.0 Volts, inclusive, but not limited thereto, can be delivered to test whether the distal tip of the spinal needle is at an appropriate location to proceed with an ablation procedure. During or following the stimulation, the patient is asked whether and where they feel the stimulation. If the stimulation is felt within the epigastric region, then it can be concluded that the distal tip of the spinal needle is at an appropriate location. If the stimulation is instead experienced in a girdle like fashion around the intercostal spaces, then the needle should be pushed anteriorly, and then the stimulation test should be performed again until the patient feels the stimulation in the epigastric region.

In accordance with certain embodiments, after the distal tip of the RF needle is positioned at an appropriate location for performing splanchnic nerve ablation, a local anesthetic is injected through the RF needle to anesthetize the nerves that are to be ablated. The local anesthetic that is used to anesthetize the nerves that are to be ablated can be, e.g., lidocaine (having a concentration between 1% and 2%, inclusive) or ropivacaine (having a concentration between 0.25% and 0.5%, inclusive), but is not limited thereto. Ablation can then be performed starting about 1 to 2 minutes after the local anesthetic is delivered.

It may take on the order of about 24 to 72 hours after a splanchnic nerve ablation procedure is performed for the beneficial effects of the splanchnic nerve ablation (i.e., a reduction in dysmotility symptoms) to be realized by the patient. In accordance with certain embodiments, in order to provide more immediate dysmotility relief to the patient, after the splanchnic nerve ablation is performed, the same needle used to perform the ablation can be used to deliver a local anesthetic to at least one of the splanchnic nerves. For example, the local anesthetic can be bupivacaine (having a concentration between 0.25% and 0.75%, inclusive), chloroprocaine (having a concentration between 1% and 2%, inclusive), mepivacaine (having a concentration between 0.25% and 0.5%, inclusive), ropivacaine (having a concentration between 0.25% and 0.5%, inclusive), or lidocaine (having a concentration between 1% and 2%, inclusive), or combinations thereof.

As noted above, since the embodiments described above with reference to FIG. 4 utilize RF ablation, such embodiments can more specifically be referred to as splanchnic nerve radiofrequency ablation. The use of other ablation techniques are also possible, and within embodiments of the present invention. For example, instead of using radiofrequency ablation to ablate at least one of the greater, lesser or least splanchnic nerves of a patient having dysmotility, alternative ablation techniques can be used to ablate at least one of the greater, lesser or least splanchnic nerves of a patient having dysmotility. Exemplary alternative ablation techniques include, but are not limited to, electromagnetic ablation, ultrasound ablation, microwave ablation, infrared light ablation, hot water ablation and cryogenic cooling ablation. Microwave ablation, for example, is similar to RF ablation, but uses microwave frequencies, which are higher than RF frequencies, to destroy target tissue. Ultrasound ablation applies high-intensity focused ultrasound energy to locally heat and destroy target tissue. Cryogenic cooling ablation, which is also known as cryoablation, utilizes a hollow needle (also known as a cryoprobe) through which cooled, thermally conductive, fluids are circulated. More specifically, a cryoprobe is inserted into or placed adjacent to target tissue and a cryogenic freezing unit removes heat (“cools”) from the tip of the probe and by extension from the surrounding tissues.

In accordance with certain embodiments, between steps 404 and 406, a non-ionic radio-opaque contrast can be injected through the needle to check that the needle is extravascular and has not punctured a pleural cavity. If it is determined that the needle is positioned intravascularly, or has punctured a pleural cavity, the distal end of the needle should be repositioned and retested using the contrast before the needle is used to perform splanchnic nerve ablation. In the rare situation where a needle punctures one of the patient's two pleural cavities, the ablation should not be performed on the other side of the spine, to complete the bilateral splanchnic nerve ablation, until after the punctured plural cavity has had time to heal, because bilateral pneumothorax can be life threatening.

### Kits to Treat Dysmotility

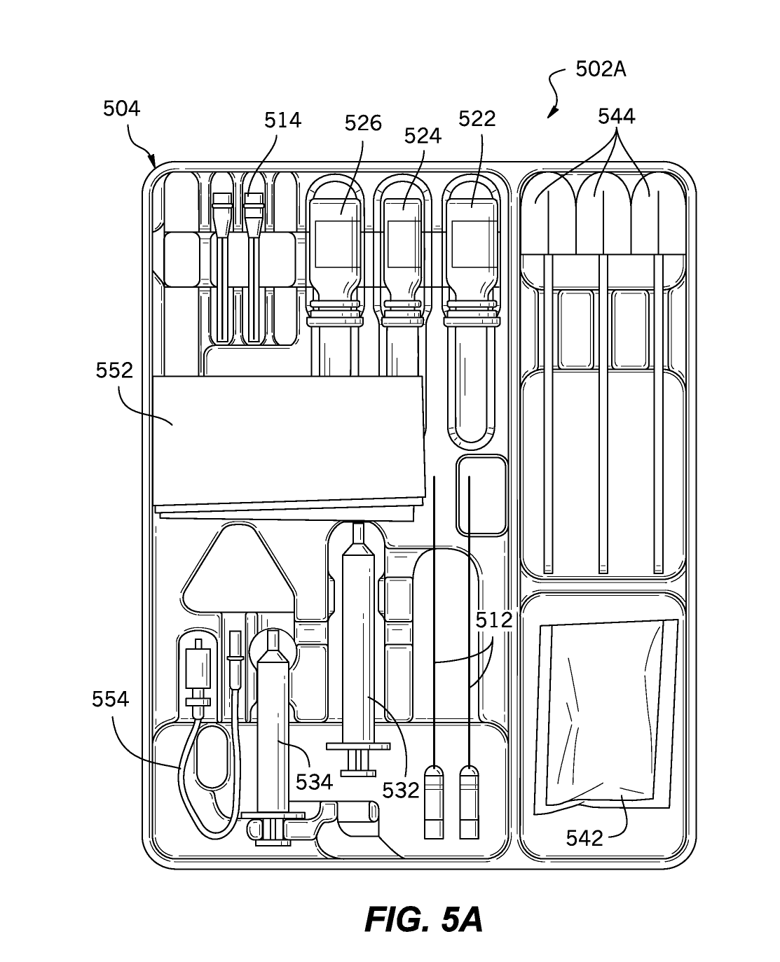

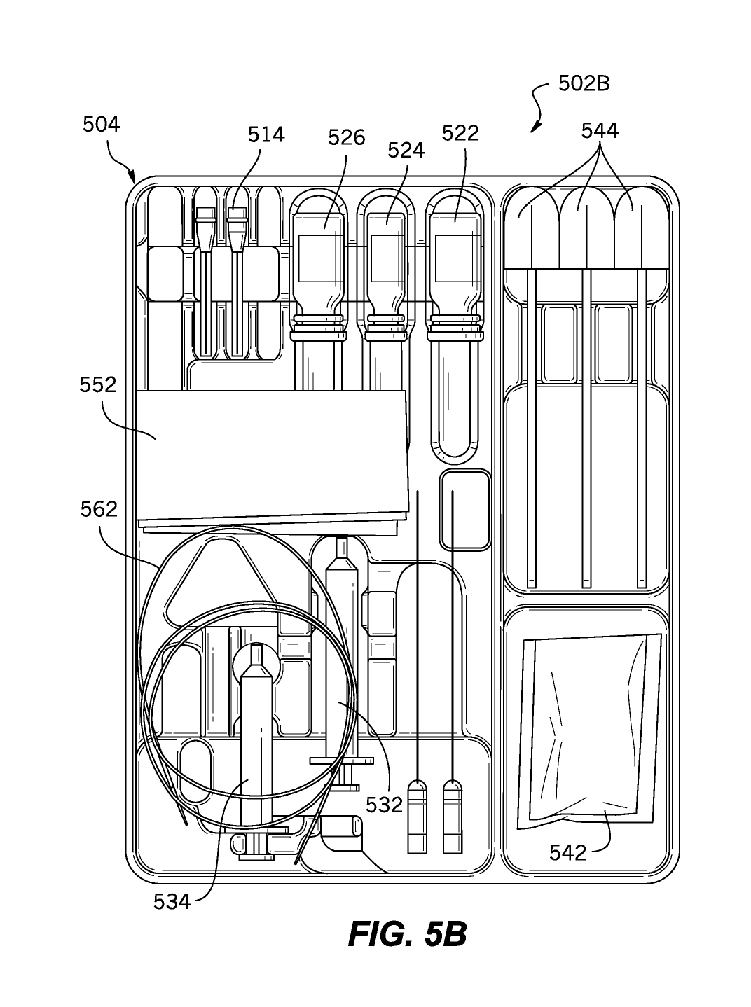

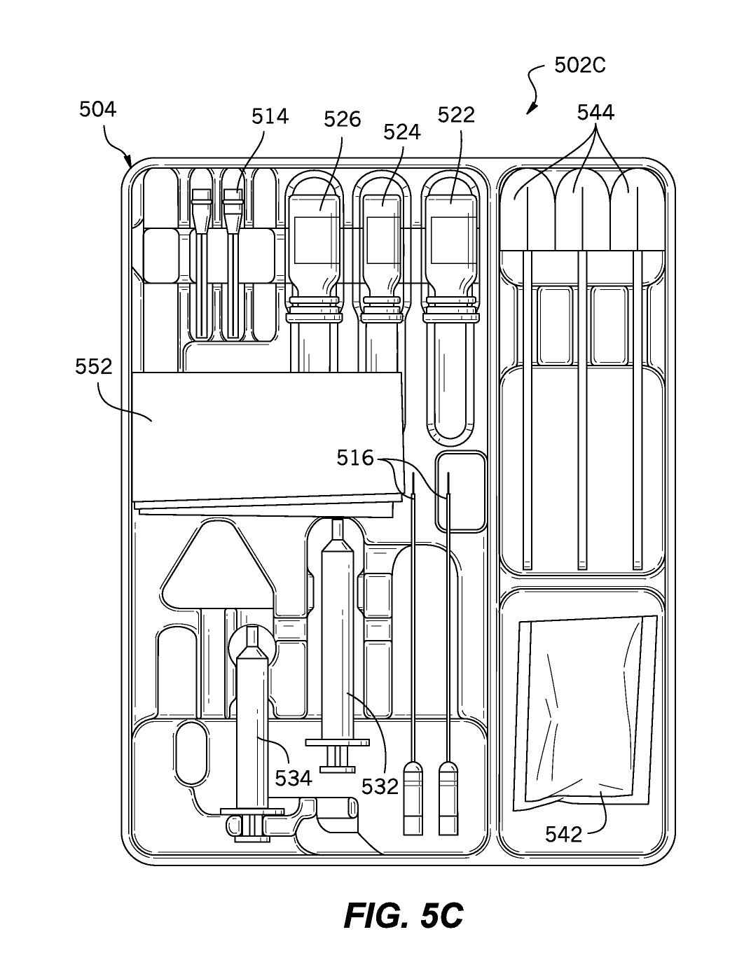

Certain embodiments of the present invention are directed to kits for use in performing splanchnic nerve block, celiac plexus block and/or splanchnic nerve ablation to treat dysmotility. Examples of such kits are described below with reference to FIGS. 5A, 5B and 5C.

FIG. 5A illustrates a kit 502A for use in performing splanchnic nerve block or celiac plexus block to treat dysmotility. The kit 502A is shown as including a tray 504 including indentations configured to receive and hold in place other components of the kit. The components of the kit 502A include a pair of spinal needles 512, a subcutaneous injection needle 514, a vial 522, a vial 524, a vial 526, a syringe 532, and a syringe 534. The kit 502A is also shown as including a packet of antiseptic solution 542, e.g., a povidone-iodine solution, and three cleaning sponges 544 that are used for applying the antiseptic solution. More or less than three sponges 544 can alternatively be included. Further, the kit 502A is shown as including one or more gauze pads 552, each of which can be, e.g., 2×2 inches, or 2×4 inches, but are not limited thereto.

The vial 522 stores about 1 fluid ounce (i.e., about 30 cubic centimeters, or about 30 milliliters) of the local anesthetic that is to be delivered, using the pair of spinal needles 506, to at least one of the greater, lesser or least splanchnic nerves of a patient, or to the celiac plexus of a patient. The local anesthetic stored in the vail 512 can be bupivacaine (having a concentration between 0.25% and 0.75%, inclusive), chloroprocaine (having a concentration between 1% and 2%, inclusive), mepivacaine (having a concentration between 0.25% and 0.5%, inclusive), ropivacaine (having a concentration between 0.25% and 0.5%, inclusive), or lidocaine (having a concentration between 1% and 2%, inclusive), or combinations thereof.

The spinal needles 512 are used to deliver the local anesthetic, stored in the vial 522, to at least one of the greater, lesser or least splanchnic nerves of a patient having dysmotility, or to the celiac plexus of the patient having dysmotility, to thereby reduce symptoms of dysmotility. Accordingly, the spinal needles 512 should be long enough such that after they are inserted through a patient's skin a distal end of the need can reach the splanchnic nerves or the celiac plexus of the patient. More specifically, in accordance with certain embodiments, each of the spinal needles 512 has a length between 5 and 7 inches. The spinal needles 512 have a hollow lumen through which a local anesthetic can be injected to deliver the local anesthetic to at least one of the greater, lesser or least splanchnic nerves (to perform splanchnic nerve block) or to the celiac plexus (to perform celiac plexus block). To accommodate a hollow lumen of sufficient diameter, an outer diameter of each of the spinal needles 512 is between 18 and 25 gauge, inclusive. Two of the spinal needles 512 are included in the kit 502A so that the splanchnic nerve block or the celiac plexus block can be perform bilaterally, as explained above. The distal tip of the spinal needles 512 can be sharp, or alternatively, can be blunt. Sharp needles more easily penetrate the skin, but are more likely to unintentionally pierce a vessel near the vertebral body T11, T12 or L1, Blunt needle are more difficult to penetrate the patient's skin, but less likely to unintentionally piece a vessel near the vertebral body T11, T12 or L1.

The syringe 532 is used to inject the local anesthetic through the spinal needles 522. Accordingly, the syringe 532 is configured to be connected to the spinal needles 512. The syringe 532 can be a 10 cc (cubic centimeter) syringe, but is not limited thereto.

The vial 524 stores the local anesthetic (e.g., lidocaine having a concentration between 0.5% and 2%, inclusive) used to anesthetize (i.e., numb) the skin and create skin wheals, prior to the spinal needles 512 being injected through the skin.

The subcutaneous injection needle 514 is used to deliver the local anesthetic, stored in the vial 524, just below the skin, and thus, can have a length between 1 and 2 inches, inclusive, and a gauge between 22 and 27, inclusive. An additional needle, that has a smaller gauge (and thus, a larger diameter) than the subcutaneous injection needle 514, and is shown to the left of the subcutaneous injection needle 514, can be included in the kit for use in expediting filling of the syringe(s) 532 and/or 534 with contents of one or more of the vials described above. Such a filling needle can have a gauge, e.g., between 16 and 22, inclusive, but is not limited thereto.

The syringe 534 is used to inject the local anesthetic (stored in the vial 524) through the injection needle 514. Accordingly, the syringe 534 is configured to be connected to the injection needle 514. The syringe 534 can be a 5 cc (cubic centimeter) syringe, but is not limited thereto.

The vial 526 stores about 0.33 fluid ounces (i.e., about 10 cubic centimeters, or about 10 milliliters) of a saline solution that can be used to selectively dilute one or both of the local anesthetics that are stored in the vials 512 and 514. The saline solution can, for example, be sodium chloride having a concentration of about 0.9%, but is not limited thereto.

The gauze pad(s) 552 can be used, e.g., to clean away blood from the sites where the spinal needles 512 are injected, e.g., if one of the spinal needles accidentally pierces a blood vessel.

The kit 502B is also shown as including a length of clear tubing 554 that can be used to facilitate injection of radio-opaque contrast and/or local anesthetic via the spinal needles 512. For example, the clear tubing 554 can be connected between the distal end of the syringe 532 and the proximal end of one of the spinal needles 512. Accordingly, one end of the clear tubing 554 can be configured to connect to a distal end of the syringe 532 and the other end of the tubing 554 can be configured to connect to a proximal end of a spinal needle 512.

FIG. 5B illustrates an alternative kit 502B for use in performing splanchnic nerve block or celiac plexus block to treat dysmotility. The kit 502B is substantially the same as the kit 502A, except that the kit 502B also includes a catheter 562. The components in the kit 502B that are the same as those in the kit 502A are labeled the same, and need not be described again. The catheter 562 is for use in threading through each of the spinal needles 512 and delivering portions of the second local anesthetic (stored in the vial 522) to at least one of the greater, lesser or least splanchnic nerves or a celiac plexus of a patient. The catheter 562 has a length that is greater than the length of each of the spinal needles 512, so that while the catheter 562 is threaded through one of the spinal needles 512, a proximal end of the catheter can extend from a proximal end of the spinal needle 512 (and be attached to the syringe 532) while a distal end of the catheter 562 extends from a distal end of the spinal needle 512. The length of the catheter 562 is preferably at least twice the length of each of the spinal needles 562, or can be shorter, but is preferably at least 4 inches greater than the length of each of the spinal needles 562. An outer diameter of the catheter 512 should be smaller than an inner diameter of the spinal needles 512 so that the catheter 562 can be threaded through each of the spinal needles 512. Where the catheter 562 is included in the kit 502B, the syringe 532 should be configured to connect to the distal end of the catheter. The kit 502B can include a single catheter 562 for use with both spinal needles 512, or a pair of catheters 562 so that there is a separate catheter 562 for use with each separate spinal needle 512. Where the catheter 562 is used to deliver a local anesthetic to at least one of the greater, lesser or least splanchnic nerves of a patient having dysmotility, or to the celiac plexus of the patient having dysmotility, in certain instances the catheter 562, after being threaded through a single one of the spinal needles 512, may be maneuvered to achieve bilateral block without having to utilize the other one of the spinal needles 512.

Additional details for how to utilize the kits 502A and 502B can be appreciated from the above description of FIGS. 1-4, and more specifically, the embodiments described with reference to FIGS. 2 and 3.

FIG. 5C illustrates a kit 502C for use in performing splanchnic nerve ablation to treat dysmotility. The kit 502C has many of the same elements as the kit 502A, except that the kit 502C includes a pair of active tip radiofrequency (RF) needles 516, instead of the pair of spinal needles 512. The components in the kit 502C that are the same as those in the kit 502A are labeled the same, and need not be described again. In accordance with specific embodiments, a length of each of the active tip RF needles is between 5 and 7 inches, inclusive, and the outer diameter is between 18 and 25 gauge, inclusive. An outer surface of each the active tip RF needles is insulated except an uninsulated distal portion that is between 0.25 and 0.75 inches in length. Additional details for how to utilize the kit 502C can be appreciated from the above description of FIGS. 1-4, and more specifically, the embodiments described with reference to FIG. 4.

Each of the kits 502A, 502B and 502C described above can also include a sterile bag or other sterile container in which the tray 504 and the components in the tray 504 can be placed to be kept sterile until the kit is used.

Although the foregoing invention has been described in some detail by way of illustration and example, for purposes of clarity and understanding, it will be obvious that various alternatives, modifications and equivalents may be used and the above description should not be taken as limiting in scope of the invention. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method for treating dysmotility, comprising:
delivering a local anesthetic to at least one of the greater, lesser or least splanchnic nerves of a patient having dysmotility, or to the celiac plexus of the patient having dysmotility, to thereby reduce symptoms of dysmotility.

2. The method of claim 1, wherein the local anesthetic is delivered bilaterally to the at least one of the greater, lesser or least splanchnic nerves of the patient to achieve bilateral splanchnic nerve block, to thereby reduce symptoms of dysmotility.

3. The method of claim 2, wherein the local anesthetic is delivered to the at least one of the greater, lesser or least splanchnic nerves to thereby interrupt efferent sympathetic fiber transmission from the splanchnic nerves of the patient to the spinal cord and brain of the patient.

4. The method of claim 1, wherein the local anesthetic includes at least one of bupivacaine, chloroprocaine, mepivacaine, ropivacaine, or lidocaine.

5. The method of claim 4, wherein the local anesthetic includes bupivacaine and has a concentration of bupivacaine between 0.25% and 0.75%, inclusive.

6. The method of claim 1, further comprising:
inserting a portion of a needle into the patient having dysmotility; and
positioning a distal end of the needle adjacent to at least one of the thoracic vertebral segment numbers 11 or 12 (T11 or T12) of the patient;
wherein the delivering comprises, while the distal end of the needle is positioned adjacent to the at least one of the T11 or T12 of the patient, using the needle to deliver the local anesthetic to at least one of the greater, lesser or least splanchnic nerves of the patient, to thereby reduce symptoms of dysmotility.

7. The method of claim 1, further comprising:
inserting a portion of a needle into the patient having dysmotility;
positioning a distal end of the needle adjacent to the L1 lumbar vertebrae of the patient; and
inserting a catheter through the needle and passing a distal end of the catheter past the distal end of the needle so that the distal end of the catheter is adjacent to least one of the thoracic vertebral segment numbers 11 or 12 (T11 or T12) of the patient;
wherein the delivering comprises, while the distal end of the catheter is positioned adjacent to the at least one of the T11 or T12 of the patient, using the catheter to deliver the local anesthetic to the one of the greater, lesser or least splanchnic nerves of the patient, to thereby reduce symptoms of dysmotility.

8. The method of claim 1, wherein the local anesthetic is delivered bilaterally to the celiac plexus of a patient of to achieve bilateral celiac plexus block, to thereby reduce symptoms of dysmotility.

9. The method of claim 8, wherein the local anesthetic is delivered to the celiac plexus of the patient to interrupt efferent sympathetic fiber transmission from the celiac plexus of the patient to the splanchnic nerves, spinal cord and brain of the patient.

10. The method of claim 1, further comprising:
inserting a portion of a needle into the patient having dysmotility; and
positioning a distal end of the needle adjacent to the L1 lumbar vertebrae of the patient;
wherein the delivering comprises, while the distal end of the needle is positioned adjacent to the L1 lumbar vertebrae of the patient, using the needle to deliver the local anesthetic to the celiac plexus of the patient, to thereby reduce symptoms of dysmotility.

11. The method of claim 1, further comprising:
inserting a portion of a needle into the patient having dysmotility;
positioning a distal end of the needle posterior to and between a mid to anterior portion of the L1 lumbar vertebra of the patient; and
inserting a catheter through the needle and passing a distal end of the catheter past the distal end of the needle so that the distal end of the catheter is adjacent to the L1 lumbar vertebrae of the patient;
wherein the delivering comprises, while the distal end of the catheter is positioned adjacent to the L1 lumbar vertebrae of the patient, using the catheter to deliver the local anesthetic to the celiac plexus of the patient, to thereby reduce symptoms of dysmotility.

12. A method for treating dysmotility, comprising:
ablating a portion of at least one of the greater, lesser or least splanchnic nerves of a patient having dysmotility, to thereby reduce symptoms of dysmotility.

13. The method of claim 12, wherein the ablating is performed bilaterally.

14. The method of claim 12, wherein the ablating is performed using radiofrequency ablation.

15. The method of claim 12, further comprising:
inserting a portion of a needle into the patient having dysmotility, wherein a proximal portion of the needle is insulated and a distal end portion of the needle is uninsulated; and
positioning the uninsulated distal end portion of the needle adjacent to at least one of the thoracic vertebral segment numbers 11 or 12 (T11 or T12) of the patient;
wherein the ablating comprises, while the uninsulated distal end portion of the needle is positioned adjacent to the at least one of the T11 or T12 of the patient, using the uninsulated distal end portion of the needle to deliver an alternating radiofrequency (RF) current to the at least one of the greater, lesser or least splanchnic nerves of the patient having dysmotility.

16. The method of claim 15, wherein a frequency of the alternating RF current is within a range of 450 kHz to 1200 kHz, inclusive.

17. The method of claim 12, wherein the ablating is performing using one of the following ablation techniques:
electromagnetic ablation;
ultrasound ablation;
microwave ablation;
infrared light ablation;
hot water ablation; or
cryogenic cooling ablation.

18. A kit for use in performing bilateral splanchnic nerve block or celiac plexus block to treat dysmotility, the kit comprising:
a pair of spinal needles each having a length between 5 and 7 inches, inclusive, and an outer diameter between 18 and 25 gauge, inclusive;
a first vial storing a first local anesthetic for use in anesthetizing a patient's skin;
an injection needle having a length between 1 and 2 inches,
inclusive, and an outer diameter between 22 and 27 gauge, inclusive, the injection needle for use in injecting portions of the first local anesthetic at sites where the spinal needles are to be inserted into a patient's skin;
a first syringe for use in injecting portions of the first local anesthetic through the injection needle;
a second vial storing a second local anesthetic that is to be delivered, using the pair of spinal needles, to at least one of the greater, lesser or least splanchnic nerves of a patient or to the celiac plexus of a patient; and
a second syringe for use in injecting portions of the second local anesthetic through the pair of spinal needles.

19. The kit of claim 18, wherein the second syringe is configured to connect to distal ends of the pair of spinal needles.

20. The kit of claim 18, further comprising:
a catheter for use in threading through each of the pair of spinal needles and delivering portions of the second local anesthetic to at least one of the greater, lesser or least splanchnic nerves or the celiac plexus of a patient;
wherein the catheter has a length that is greater than the length of each of the spinal needles, so that while the catheter is threaded through one of the spinal needles, a proximal end of the catheter can extend from a proximal end of the one of the spinal needles while a distal end of the catheter extends from a distal end of the one of the spinal needles;
wherein an outer diameter of the catheter is smaller than an inner diameter of each of the spinal needles so that the catheter can be threaded through each of the spinal needles; and
wherein the second syringe is configured to connect to the distal end of the catheter.

21. The kit of claim 18, wherein the second local anesthetic comprises at least one of bupivacaine, chloroprocaine, mepivacaine, ropivacaine or lidocaine.

22. The kit of claim 19, further comprising an antiseptic and one or more sponges for use in cleaning portions of a patient's skin prior to the needles being injected therein.

23. The kit of claim 18, further comprising:
a third vial storing a saline solution for use in diluting a concentration of at least one of the first and second local anesthetics.

24. A kit for use in performing bilateral splanchnic nerve ablation to treat dysmotility, the kit comprising:
a pair of active tip radiofrequency (RF) needles each having a length between 5 and 7 inches, inclusive, and an outer diameter between 18 and 25 gauge, inclusive, wherein an outer surface of each the RF needles is insulated except for an uninsulated distal portion that is between 0.25 and 0.75 inches in length;
a first vial storing a first local anesthetic for use in anesthetizing a patient's skin and/or anesthetizing at least one of the greater, lesser or least splanchnic nerves of a patient prior to RF ablation thereof;
an injection needle having a length between 1 and 2 inches, inclusive, and an outer diameter between 22 and 27 gauge, inclusive, the injection needle for use in injecting portions of the first local anesthetic at sites where the RF needles are to be inserted into a patient's skin; and
a first syringe for use in injecting portions of the first local anesthetic through the injection needle.

25. The kit of claim 24, further comprising:
a second vial storing a second local anesthetic that can be delivered, using the active tip RF needles, to at least one of the greater, lesser or least splanchnic nerves of a patient after at least one of the greater, lesser or least splanchnic nerves have been ablated using the active tip RF needles;
wherein the second local anesthetic comprises at least one of bupivacaine, chloroprocaine, mepivacaine, ropivacaine or lidocaine.

26. The kit of claim 25, further comprising:
an antiseptic and one or more sponges for use in cleaning portions of a patient's skin prior to the needles being injected therein; and
a third vial storing a saline solution for use in diluting a concentration of at least one of the first and second local anesthetics.
